(19) United States
(12) Patent Application Publication
(10) Pub. No.: US 2013/0344174 A1
(43) Pub. Date: Dec. 26, 2013

(54) Bioactive Fractions and Compounds from Polygonum genus, their Use in Anti-diarrhea and the Method of Preparation

(71) Applicant: HONG KONG BAPTIST UNIVERSITY, Hong Kong (HK)

(72) Inventors: Zhaoxiang BIAN, Hong Kong (HK); Haitao XIAO, Hong Kong (HK); Shilin CHEN, Hong Kong (HK); Dajian YANG, Hong Kong (HK); Hongxi XU, Hong Kong (HK); Aiping LU, Hong Kong (HK); Albert Sun-Chi CHAN, Hong Kong (HK)

(73) Assignee: HONG KONG BAPTIST UNIVERSITY, Hong Kong (HK)

(21) Appl. No.: 13/831,899

(22) Filed: Mar. 15, 2013

Related U.S. Application Data
(60) Provisional application No. 61/662,354, filed on Jun. 21, 2012.

(51) Int. Cl. A61K 36/704; A61K 31/7048; A61K 31/366

(52) U.S. Cl. CPC A61K 36/704; A61K 31/366; A61K 31/7048
USPC 424/725; 549/278; 536/4.1; 514/453; 514/25

(57) ABSTRACT

The present invention discloses the utility of factions and compounds from Polygonum chinense Linn in anti-diarrhea. The invention also includes their preparation and application method for anti-diarrhea treatment.

### CROSS-REFERENCE TO RELATED APPLICATIONS

The present application claims priority of U.S. provisional application No. 61/662,354 filed Jun. 21, 2012, and which the disclosure is hereby incorporated by reference by its entirety.

### FIELD OF INVENTION

The present invention relates to the therapeutic applications of fractions and compounds from Polygonum chinense Linn in anti-diarrhea. The present invention also includes preparation and application method for anti-diarrhea treatment.

### BACKGROUND OF INVENTION

Diarrhea is a common gastrointestinal disorder characterized by an increase in stool frequency and a change in stool consistency. It can be fatal and claims the lives of 3-4 million infants and children worldwide every year, it also happens to be amongst the symptoms of many other diseases. The plausible implicating factors inducing diarrhea can range from infective to immunological and nutritive factors. Although nutritive supplements have proved beneficial in acute diarrhea, chronic diarrhea still remains elusive and can often culminate in serious effects if left untreated. In order to defend oneself from the onslaught of diarrhea, it would be therefore worthwhile to use natural products as the propensity of adverse effects with chemical drugs is high. Heinrich et al. have reported that despite the availability of simple and cheap treatments for diarrhea, healers and patients in many communities still rely on locally available phytomedicines. It is well known that plants have long been a very important source of new drugs. Many plant species have been screened for substances with therapeutic activity. Medicinal plants are a promising source of anti-diarrheal drugs. For this reason, international organizations including the World Health Organization (WHO) have encouraged studies pertaining to the treatment and prevention of diarrhea diseases using traditional medical practices. These formed the basis of researches on Chinese medicinal plants with anti-diarrhea activity.

Polygonum chinense Linn, a perennial herb, belongs to the family of Polygonaceae, is mainly distributed in southwestern regions of China and commonly consumed for treatment of enteritis. There has been no report on anti-diarrhea activity of P. chinense.

Citation or identification of any reference in this section or any other section of this application shall not be construed as an admission that such reference is available as prior art for the present application.

### SUMMARY OF INVENTION

The present application discloses the novel effective anti-diarrhea activity in P. chinense. The objective of this invention is to provide new anti-diarrhea treatment by phytotherapy.

In accordance with one aspect of the present invention, there is provided a method for extraction and separation of fractions and compounds from a natural source for use in anti-diarrhea treatment.

In another aspect of the present invention, there is provided a treatment of diarrhea related diseases, which comprises administering a composition comprising a therapeutic effective amount of bioactive fractions, compounds from P. chinense, or a mixture thereof.

Those skilled in the art will appreciate that the invention described herein is susceptible to variations and modifications other than those specifically described.

The invention includes all such variation and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows characteristics of various fractions of P. chinense under UPLC. FIG. 1A is UPLC chromatography of TF25(254 nm); FIG. 1B is UPLC chromatography of TF50 (254 nm); FIG. 1C is MS and MS/MS spectra of peak at the retention time of 7.815 min in UPLC chromatography of TF25; and FIG. 1D is MS and MS/MS spectra of peak at the retention time of 10.219 min UPLC chromatography of TF25.

FIG. 2 shows the stools rate, stools grade and diarrhea index of castor oil or magnesium sulfate-induced diarrhea in mice having treated with ethanol extract of P. chinense (EE) (n=9).

FIG. 3 are HPLC chromatography profiles of standard markers corilagin (FIG. 3A) and ellagic acid (FIG. 3B); and the ethanol extract of P. chinense (EE; FIG. 3C).

FIG. 4 shows the stools rate, stools grade and diarrhea index of castor oil or magnesium sulfate-induced diarrhea in mice having treated with various fractions from the ethanol extract of P. chinense (EE) (n=9).

FIG. 5 shows the stools rate, stools grade and diarrhea index of castor oil and magnesium sulfate-induced diarrhea in mice having treated with subfractions from the combination of BF and AF (TF) (n=8−9).

FIG. 6 shows the stools rate, stools grade and diarrhea index of castor oil or magnesium sulfate-induced diarrhea in mice having treated with ellagic acid or corilagin (n=9−10).

FIG. 7 shows antienteropooling and intestinal transit activities of ellagic acid and corilagin in mice (n=8−9).

### DETAILED DESCRIPTION OF INVENTION

Reference is made in detail to the presently preferred embodiment of the present invention, which serves to explain the principles of the invention. The embodiments or examples disclosed herein are described in sufficient detail to enable those skilled in the art to practice the invention, and it is to be understood that other embodiments may be utilized, and that changes may be made without departing from the spirit of the present invention.

The present invention pertains to a composition for anti-diarrhea treatment and/or related diseases and method of preparation thereof. The composition comprises_a therapeutic effective amount of bioactive fractions, compounds or extract from a herb from the family of Polygonaceae, a compound of formula (1) or (2), salts, derivatives thereof, or a combination thereof. The chemical structures of compound of formula (1) and (2) are as follows:

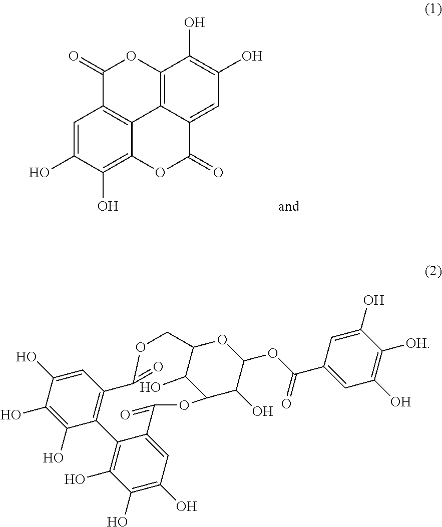 [SMILES: O=c1oc2c(O)c(O)cc3c(=O)oc4c(O)c(O)cc1c4c23 ; "and" ; O=C(OC1OC2COC(=O)c3cc(O)c(O)c(O)c3-c3c(cc(O)c(O)c3O)C(=O)OC(C2O)C1O)c1cc(O)c(O)c(O)c1]

wherein compounds of formula (1) and (2) are ellagic acid and corilagin, respectively.

In particular, the present composition is effective in treatment of diarrhea or related disease by oral administration thereof. The subject in need thereof is a mammal, particularly a human._In one embodiment, where the composition is bioactive fractions, compounds or extract from a herb of Polygonaceae family, the composition is orally administered to a subject in need thereof at a dosage of no less than 1.117 gram/kilogram body weight/day. In an embodiment, the dosage is 2.355 or 4.710 gram/kilogram body weight/day.

Where the compound is formula (1), the composition is orally administered to the subject in need thereof at a dosage of no less than 15 milligram/kilogram body weight/day. In another embodiment, the dosage is 30 milligram/kilogram body weight/day.

Where the compound is formula (2), the composition is orally administered to the subject in need thereof at a dosage of no less than 30 milligram/kilogram body weight/day. In yet another embodiment, the dosage is 60 milligram/kilogram body weight/day.

In one embodiment, the_bioactive fractions, compounds or extract from a herb from the family of Polygonaceae, a compound of formula (1) or (2), salts, derivatives thereof are obtained from Polygonum chinense Linn.

The present invention also pertains to method of preparing bioactive fractions or compounds from a herb of Polyoneceae family, particularly from Polygonum chinense Linn. The bioactive fractions or compounds prepared and obtained according to the present invention is useful for anti-diarrhea treatment and/or related diseases.

The method comprises obtaining a viscous extract from dried aerial part of the Polyoneceae family herb and partitioning the viscous extract using at least one solvent to obtained fractions thereof. Examples of solvent useful in partitioning plant extract include, but are not limited to, petroleum ether (PE), ethyl acetate (EF) and n-butanol (BF).

In one embodiment, the viscous extract is partitioned with PE, EF and BF to obtain Polyoneceae fractions of PE, EF, BF and aqueous fraction thereof. In one embodiment, where the herb is P. chinense, the BF and aqueous fractions are combined, further filtered and evaporated by conventional evaporator forming a combination fraction. One skilled in the art would readily use different solvents for partitions and combine different fractions thereof to form various combination fractions for said further filtration and evaporation.

The method further comprises fractioning the combination fraction in absorption column readily used in the art. The combination fraction is eluted by deionized water and desorbed with ethanol solution. The desorbing ethanol solution may be 25%, 50% or 95%. The eluted ethanol fraction is further concentrated and vaccum-dried to produce bioactive fractions or compounds of the Polyoneceae herb for anti-diarrhea treatment.

Examples of diarrhea related diseases include, but are not limited to, enteritis, dysentery, inflammatory bowel disease, irritable bowel syndrome, irritable pouch syndrome.

### MATERIALS AND METHODS

Plant Materials and Reagents

The aerial part of P. chinense was collected from Guangdong province, China, in July 2010. The herb was authenticated by Dr. Chen Hu-biao (School of Chinese Medicine, Hong Kong Baptist University, Hong Kong, China). A voucher specimen was stored at School of Chinese Medicine, Hong Kong Baptist University, Hong Kong, China (No. P100701).

Standard marker of colilagin was gifted by Dr. Han Quanbin (School of Chinese Medicine, Hong Kong Baptist University), and standard marker of ellagic acid was gifted by Dr. Yang Xian-wen (Key Laboratory of Marine Bio-resources Sustainable Utilization, South China Sea Institute of Oceanology, Chinese Academy of Sciences). Loperamide was obtained from Xian-Janssen Pharmaceutical Co. Ltd. (Xi'an, China). Acetonitrile for mobile phase and extraction solvent was of HPLC grade (Lab-scan, Bangkok, Thailand). Deionized water was generated from a milli-Q water system (Millipore, Bedford, Mass., USA). Analytical grade reagents (Huada, Guangdong, China) were used for sample preparation.

Extraction, Fractionation, Isolation and Phytochemical Analysis

Preparation and Fractionation of the EtOH-Extract

The dried aerial part of P. chinense (2.0 kg) were extracted twice with six-fold 75% EtOH for 2 h each at reflux, evaporating under reduced pressure to remove ethanol and concentrating to yield a viscous ethanol extract (382 g, yield 19.1%, w/w). The ethanol extract was further partitioned using petroleum ether (PE), ethyl acetate and n-butanol. The ethanol extract (EE), as well as petroleum ether fraction (PE, yield 9.1%, w/w), ethyl acetate fraction (EF, yield 18.7%, w/w), n-butanol fraction (BF, yield 19.6%, w/w) and remaining aqueous fraction (AF, yield 49.2%, w/w) were subjected to pharmacological study. At the time of use, tested drugs such as PE, EF, BF, AF and positive anti-diarrheal agent loperamide were freshly reconstituted in 0.5% sodium carboxymethylcellulose (CMC-Na) solution.

Based on the pharmacological study data of different solvent extractions, n-butanol fraction (BF) and aqueous fraction (AF) were combined and subjected to further fractionation on a Diaion HP-20 macroporous adsorption resins (Mitsubishi Kasei, Tokyo, Japan) column. The combined BF and AF (TF) mixture was dissolved in hot water and added slowly into the pretreated macroporous adsorption resins column. After reaching the adsorption equilibrium, resins were first eluted with deionized water and then eluted with 25%, 50% and 95% ethanol solution, consecutively. After concentration and vacuum-drying, the water fraction (TFO, yield 76.16%, w/w), the 25% ethanol fraction (TF25, yield 11.49%, w/w), the 50% ethanol fraction (TF50, yield 4.30%, w/w) and the 95% ethanol fraction (TF95, yield 1.40%, w/w) were obtained, separately. Drugs to be tested, namely TFO, TF25, TF50, TF95 and loperamide, were freshly reconstituted in 0.5% CMC-Na solution for further pharmacological study.

Phytochemical Analysis

The identification of major compounds in active fractions of P. chinense was conducted utilizing the UPLC-MS/MS technique. Chromatographic separation was performed using an Agilent 1290 Infinity UHPLC system (Santa Clara, Calif., USA), equipped with a binary solvent delivery system and a standard auto-sampler. A 100 mm×2.1 mm ACQUITY BEH C₁₈ 1.7 μm column (Waters Corp., Milford, USA) was used to separate active fractions of the ethanol extract of P. chinense. The mobile phase consisted of (A) 0.1% formic acid in water and (B) 0.1% acetic acid in acetonitrile. A linear gradient was optimized as follows (flow rate, 0.35 mL/min): 0-18 min, 2-28% B; 18-25 min, 28-80% B; 25-25.1 100% B. The injection volume was 2 mL and the column temperature was maintained at 50° C. in each run. Mass spectrometry was performed using an Agilent 6540 ultra-high definition (UHD) QTOF mass spectrometer (Santa Clara, Calif., USA), equipped with a Jet Stream electrospray ionization (ESI) source. Parameters for the Jet Stream technology are the superheated nitrogen sheath gas temperature (350° C.) and flow rate (10 L/min). ESI conditions were the following: negative ion mode, capillary 4500 V, nebulizer 30 psi, drying gas 6 L/min, gas temperature 300° C., nozzle voltage 300 V, skimmer voltage 65 V; octapoleRFpeak 600 V, fragmentor 175 V. Mass spectra were recorded across the range m/z 100-1700 with accurate mass measurement of all mass peaks. A sprayer with a reference solution was used as continuous calibration in negative ion using the following reference masses: m/z 112.9856 and 966.0007. The full-scan and MS/MS data was processed with Agilent MassHunter Workstation software (version B.02.00).

After guided isolation and purification of major principles in active fractions, these two major principles were quantitatively determined using HPLC method. Chromatography was performed on Agilent Series 1100 liquid chromatography (Agilent Technologies, Palo Alto, Calif.), equipped with a vacuum degasser, a quaternary pump, an autosampler and a DAD detector, connected to an Agilent ChemStation software. The system operated at 35° C. and a Hyperisil ODS C18 column (4.6 mm×250 mm i.d., 5 μm) was used. The sample was weighed exactly, diluted with 75% aqueous CH₃OH, and filtered through a syringe filter for subsequent HPLC-UV determination. The gradients were formed by varying the proportion of 1% aqueous acetic acid (A) and acetonitrile (B). The elution system was: 0-15 min, 10-13% B linear gradient; 15-20 min, 10-13% B linear gradient; 20-30 min, 16% B isocratic; the flow rate employed was 1.0 ml/min throughout the run.

Chromatographic Separation and Purification of Major Principles in Active Fractions

The guided isolation and purification of major components in active fractions were performed under monitoring of HPLC analysis, and major components were tracked by comparison of HPLC chromatograms of active fraction and its subfractions. Firstly, 50% ethanol fraction was subjected to column chromatographic separation on silica gel using CHCl3/MeOH (9:1→7:3) as mobile systems to get a series of eluents. After combination of the same eluents with TLC detection, five subfractions were obtained as follows: TF50 Fr. I, TF50 Fr. II, TF50 Fr. III, TF50 Fr. IV and TF50 Fr. V. Subsequently, these subfractions were subjected to HPLC analysis and one major component, namely compound 1, was recognized to be mainly contained in TF50 Fr. II. Therefore, TF50 Fr. II was further subjected to column chromatographic separation on Sephadex LH-20 using CHC13/MeOH (1:1) as eluent. After TLC detection, eluents between 8 and 17 (about 20 ml for each eluent) were combined and concentrated, and then subjected to repeated recrystallization with acetone to get compound 1. For the isolation and purification of compound 2, 25% ethanol fraction was subjected to column chromatographic separation on MCI gel CHP-20P (Mitsubishi Kasei, Tokyo, Japan). After addition of sample on the MCI gel column, the resins were first eluted with deionized water and then eluted with 10% methanol, 20% methanol, 50% methanol and 100% methanol solution, consecutively. After that, these eluents were subjected to HPLC analysis, and compound 2 was recognized to be predominantly contained in eluents of 10% methanol, and then these eluents were combined for further chromatographic separation using the PerkinElmer series 200 semi-preparative HPLC system (Perkin-Elmer, Norwalk, Conn.). The system operated at 30° C. and a YMC-Pack ODS-A semi-preparative column (10 μm, 250×10 mm) was used. The injection volume was 200-400 μL and the mobile phase flow rate was 6.0 mL/min. The elution system was acetonitrile/water (15:85) isocratic. Peaks were detected at 254 and 280 nm, and the peak at 20.1 min was collected and finally compound 2 was obtained. The chemical structures of compound 1 and 2 were identified as tannins being as ellagic acid (1) and corilagin (2) by comparison of their chromatographic behaviors with those of the standard markers.

Biological Activity Tests

Animals

ICR mice weighing 18-22 g were purchased from the Laboratory Animal Services Center, The Chinese University of Hong Kong. The animals were fed with a rodent standard diet with free access to water, and were kept in rooms maintained at 22±1° C. with a 12 h light/dark cycle following international recommendations. All experimental protocols were approved by The Animal Ethics Committees of Hong Kong Baptist University, in accordance with “Institutional Guidelines and Animal Ordinance” (Department of Health, Hong Kong Special Administrative Region).

Effects on Castor Oil-Induced Diarrhea in Mice

The castor oil-induced diarrhea model, as previously described by Zhou, et al in Zhou Gannan; Hu Zhihua; Wang Yaxian; et al (Institute of Chinese Materia Medica, China Pharmaceutical University, Nanjing); An Inquiry into Preparing Diarrhea Model of Mice and Application of Diarrhea Index[J]; Chinese Traditional and Herbal Drugs; 1994-04, was used in the present study. In brief, extracts, fractions or major components of P. chinense, and loperamide were administered orally for 5 consecutive days. On day 5, each animal was kept individually in a cage, the floor of which was lined with blotting paper, and the paper was changed every hour. 30 min after the last treatment, diarrhea was induced by oral administration of castor oil (40 mL/kg). Number of total fecal pellets and wet faeces were counted in 4 h after the administration of castor oil. The diameter of wet faeces was measured and scored in order to evaluate stool consistency as follows: 1—the diameter of wet faece is less than 1.0 cm; 2—the diameter of wet faece is between 1.0 and 2.0 cm; 3—the diameter of wet faece is between 2.0 and 3.0 cm; 4- the diameter of wet faece is more than 3.0 cm. The stool rate (SR) was calculated through comparing of the number of wet faeces and total faeces, and stool grade (SG) was evaluated according to the formula:

$$SG = \frac{1 {N}_{< 1} + 2 {N}_{1 - 2} + 3 {N}_{2 - 3} + 4 {N}_{> 3}}{{N}_{wet}}$$

where N_{wet} is the number of wet faeces; N_{<1}, N₁₋₂, N₂₋₃ and N_{>3} are the number of wet faeces whose diameter are less than 1.0 cm, between 1.0 and 2.0 cm, between 2.0 and 3.0 cm, and more than 3.0 cm, respectively.

Finally, the stool severity was evaluated by the in vivo diarrheal index (DI) and expressed as follows according to the method of Zhou, et al.

DI=SR×SG

Magnesium Sulphate-Induced Diarrhea

A protocol for castor oil-induced diarrhea was followed. In brief, diarrhea was induced by orally administration of magnesium sulphate at the dose of 3 g/kg to the animals 30 min after pretreatment with extracts, fractions or major components of P. chinense, and loperamide. All the administrations are carried out through oral route. Stool rate (SR), stool grade (SG) and diarrheal index (DI) were all evaluated.

Antienteropooling Assay

The experiment was conducted as previously described with a litter revision. Animals will be randomly divided into six groups. Vehicle control group received 0.5% CMC suspension (10 ml/kg, p.o.), the positive control group was administered loperamide at the dose of 2 mg/kg orally; the test groups were orally administered major principles ellagic acid at the doses of 15 and 30 mg/kg, and corilagin at the doses of 30 and 60 mg/kg, respectively. The whole treatment lasted for 5 consecutive days. Before the last treatment, the mice of all groups were fasted overnight. After one hour of the last treatment on day 5, all groups were given the diarrhea reagent (0.5 ml/mouse of a 20% aq. MgSO₄, orally). They were killed 30 min later and the small intestines were collected and weighed to find out the accumulation of intestinal fluid secretion evoked by MgSO₄.

Gastrointestinal Transit Test

The experiment was conducted based on methods known in the art with a litter revision. Animals will be randomly divided into six groups. Vehicle control group received 0.5% CMC suspension (10 ml/kg, p.o.), the positive control group was administered loperamide at the dose of 2 mg/kg orally; the test groups were orally administered major principles ellagic acid at the doses of 15 and 30 mg/kg, and corilagin at the doses of 30 and 60 mg/kg, respectively. The whole treatment lasted for 5 consecutive days. Before the last treatment, the mice of all groups were fasted overnight. Five minutes later of the last treatment on day 5, 0.5 ml of a 3% suspension of deactivated charcoal in 0.5% aqueous Sodium Carboxymethyl Cellulose was administered orally to each mouse. All the mice were killed by cervical translocation 30 min later and the distance travelled by the charcoal plug from pylorus to caecum was determined and expressed as a percentage of the total length of the small intestine.

Statistical Analysis

The data were presented as mean value±standard deviation (SD). Statistical significances were evaluated using one-way ANOVA, followed by Duncan's multiple range tests. GraphPad Prism 5.0 software (GraphPad Software Inc., San Diego, Calif., USA) was used for all calculations, and P<0.05 was considered statistically significant.

### RESULTS

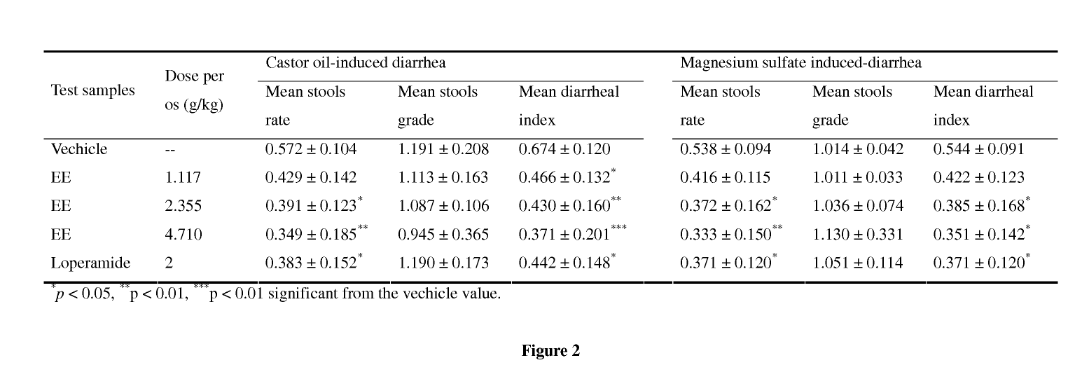

P. chinense was often used as a single herb or a major component in a formulation in treating gastrointestinal disorders such as dysentery and enteritis, with flexible ranging from 30 grams to 150 grams dependent on the severity of diseases. In the present study, three doses of the ethanol extract of P. chinense (equivalent to 30, 60 and 120 grams/60 kg human body weight/per day) were orally administered to castor oil- and magnesium sulfate-induced diarrhea mouse models for the evaluation of its anti-diarrheal activity. As shown in FIG. 2, the ethanol extract of P. chinense exhibits significant anti-diarrheal activities in a dose-dependent manner in the two models. Compared to the positive reference anti-diarrheal agent, loperamide (2 mg/kg, equivalent to 10 mg/grams/60 kg human body weight/per day), the ethanol extract of P. chinense at dose of 2.355 g/kg exhibits a comparable anti-diarrheal activity in both castor oil- and magnesium sulfate induced-diarrhea in mice, the anti-diarrheal activity of P. chinense at dose of 4.710 g/kg is better than that of loperamide (2 mg/kg).

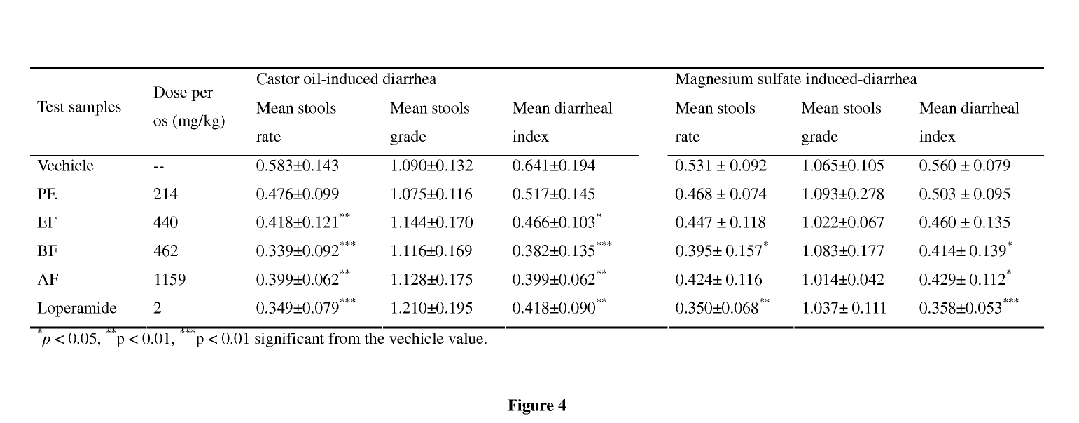

In order to identify the active components contributed to anti-diarrhea efficacy of P. chinense, a bioassay-guided fractionation and purification process was performed to elucidate its bioactive fractions and compounds. Firstly, the ethanol extract of P. chinense (EE) was submitted to subsequent solvent extractions in increasing polarity as the first step of fractionation. Each solvent extract was then administered to experimental animals in doses depending on their ratio in the original EE. The results show that petroleum ether fraction (PF) of EE was found no anti-diarrhea activity, while high polar extracts, ethyl acetate fraction (EF), n-butanol fraction (BF) and remaining aqueous fraction (AF), are significantly effective against castor oil-induced diarrhea in mice. However, the results from magnesium sulfate induced-diarrhea model is slightly different from that in castor oil-induced diarrhea model, only n-butanol fraction (BF) and remaining aqueous fraction (AF) exhibit significantly anti-diarrhea activity, while petroleum ether fraction (PF) and chloroform fraction (CF) have no obvious response (FIG. 4). These findings indicated that active fractions n-butanol fraction (BF) and remaining aqueous fraction (AF) are responsible for the observed effect of P. chinense.

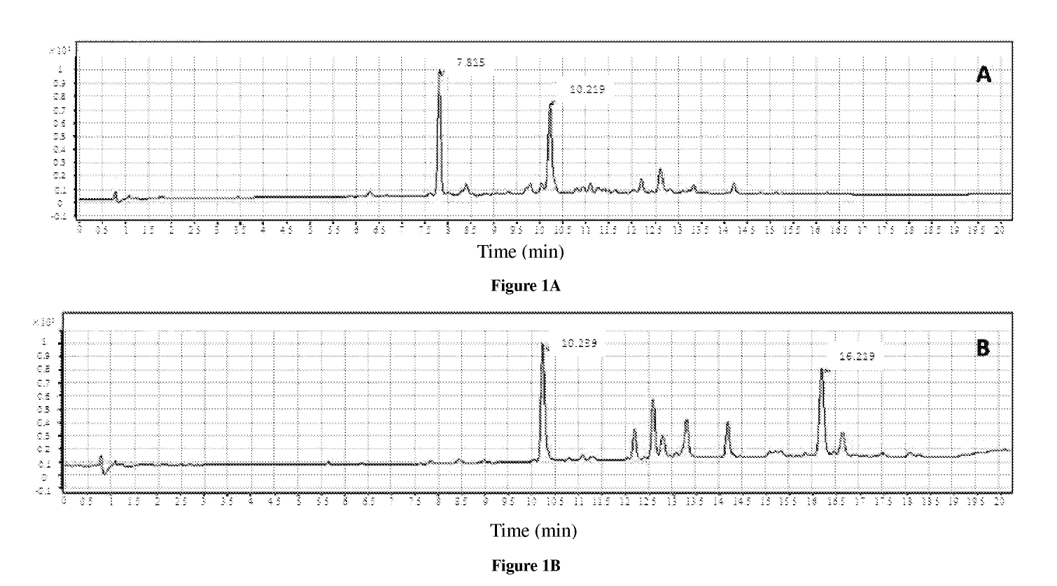

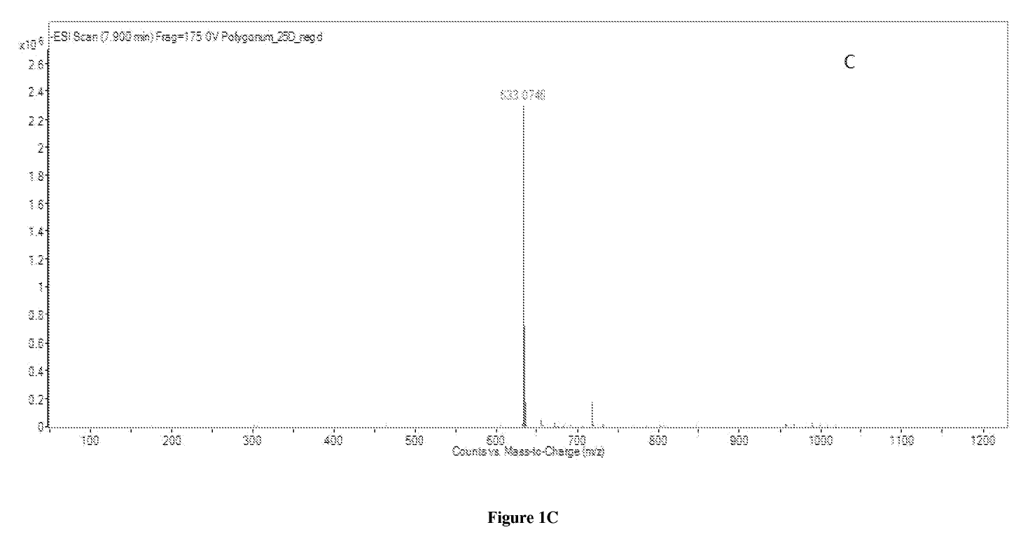

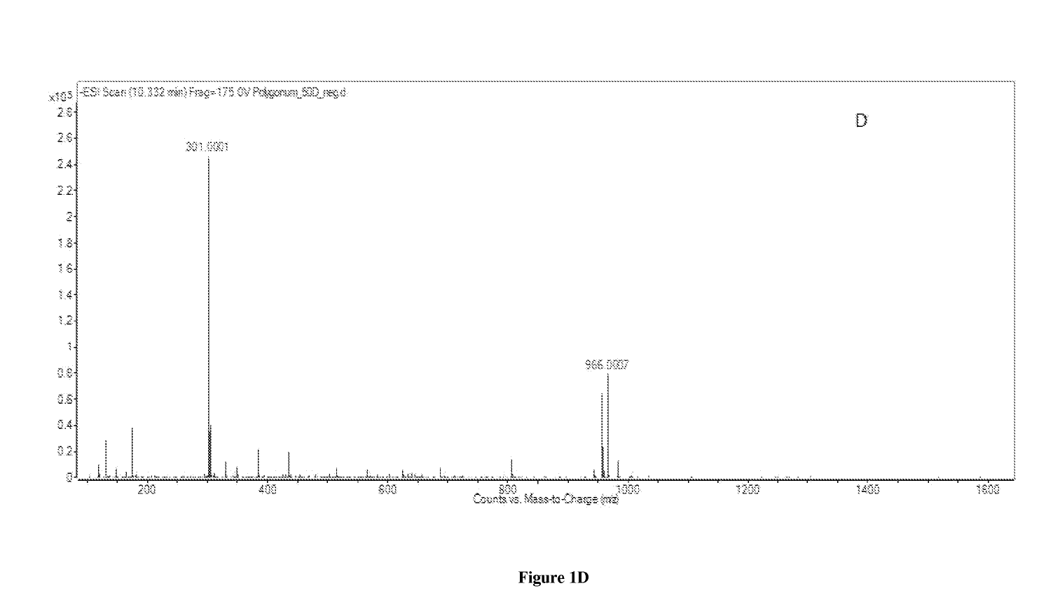

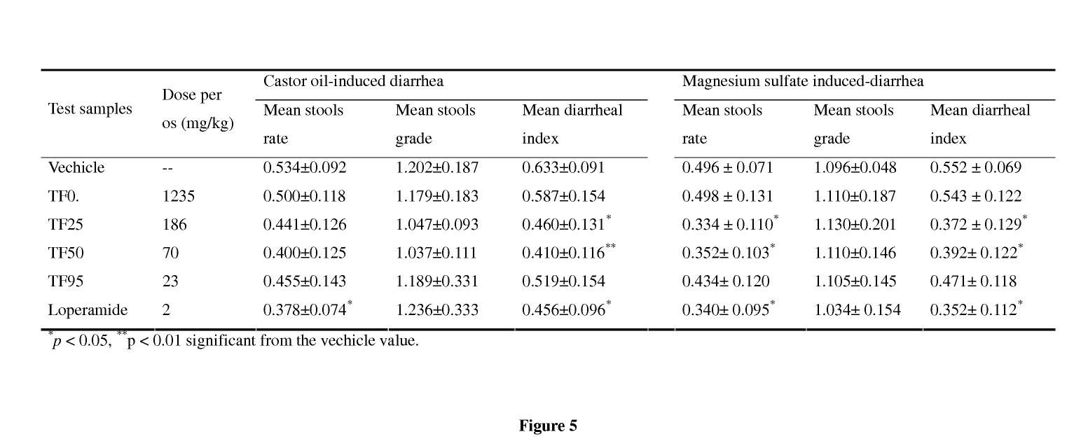

Considering BF and AF are two bordering fractions of P. chinense against diarrhea, these active fractions were therefore combined for further fractionation in order to identify the bioactive compounds. The combined BF and AF (TF) mixture was submitted to Diaion HP-20 macroporous adsorption resins column chromatography and four subfractions were obtained. The effects of these subfractions were also investigated in both castor oil- and magnesium sulphate-induced diarrhea in mice, and their doses administered to experimental animals depended on their ratio originated from the combination of BF and AF (TF). Results confirm that the 25% ethanol fraction (TF25) and the 50% ethanol fraction (TF50) have significant anti-diarrheal activities (FIG. 5). For further identification of chemical constituents of TF25 and TF50, LC-MS-MS technique was applied in order to predict their structures. After optimization of UPLC and electrospray ionization (ESI) MS-MS conditions, the UPLC-UV fingerprints of TF25 and TF50 (FIG. 1) were obtained according to the developed UPLC method described above. Tuning experiments show that the negative ion mode is more sensitive than the positive ion mode for identifying compounds in TF25 and TF50, and the UPLC-ESI-MS-MS chromatogram exhibit good consistency with that obtained from UPLC-DAD analysis. In the LC-ESI-MS-MS spectrum of TF25, two major characteristic peaks are obtained with one quasi-molecular peak [M—H]—at m/z 633.0746 and retention time at 7.815 min, and the other one with one quasi-molecular peak [M—H]—at m/z 301.0001 and retention time at 10.219 min. Compared the retention time, UV and MS spectra of above mentioned compounds with those of reference compounds, two compounds are unambiguously identified as ellagic acid (Formula 1) and corilagin (Formula 2). In addition, two major peaks at the retention time of 10.239 and 16.219 min are found on the fingerprint profile of TF50. The peak at retention time of 10.239 with a quasi-molecular peak [M—H]—at m/z 301.0364 is identified as ellagic acid (1); however, another major peak was not identified. Nevertheless, so far, no report on anti-diarrheal activity of ellagic acid (Formula 1) and corilagin (Formula 2) has been found. Thus, guided isolation and purification of ellagic acid (Formula 1) and corilagin (Formula 2) is carried out to assess their anti-diarrhea activities in animals. After isolation and purification by various chromatographic techniques, such as silica gel column, Sephadex LH-20 column, repeated recrystallization and preparative HPLC system, those two entities are finally obtained.

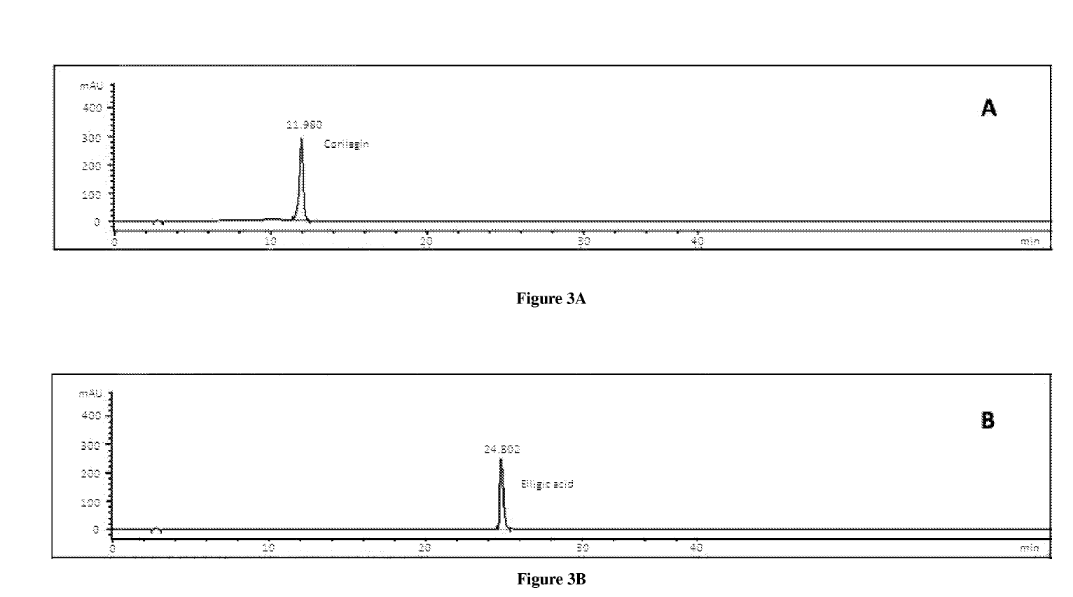

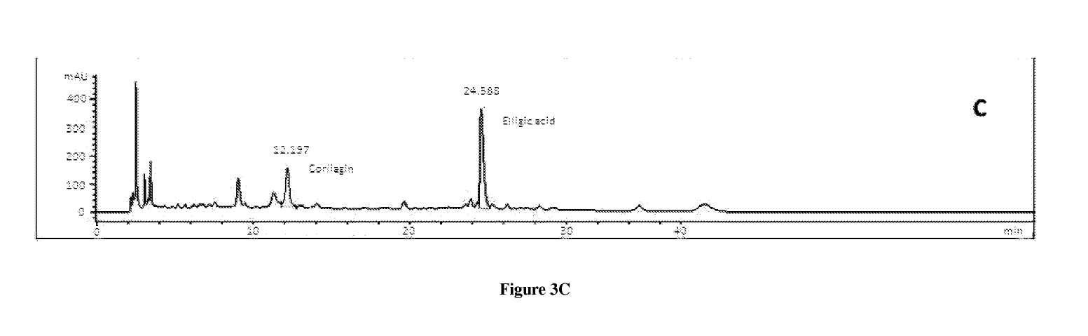

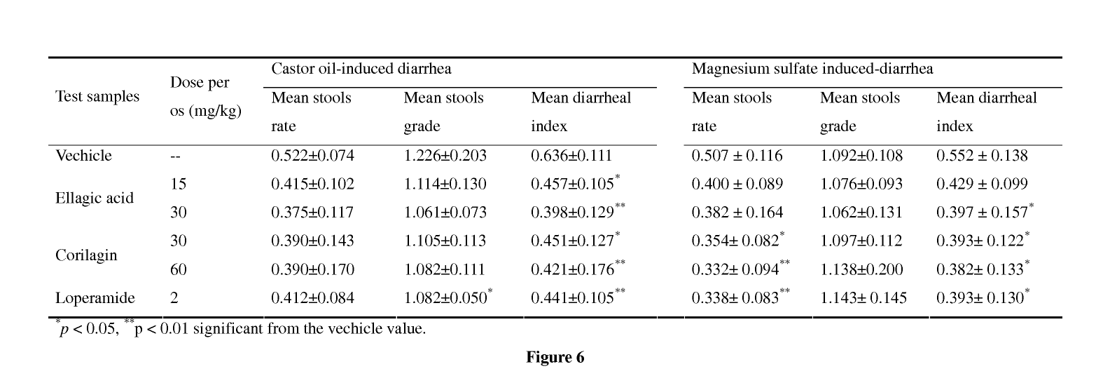

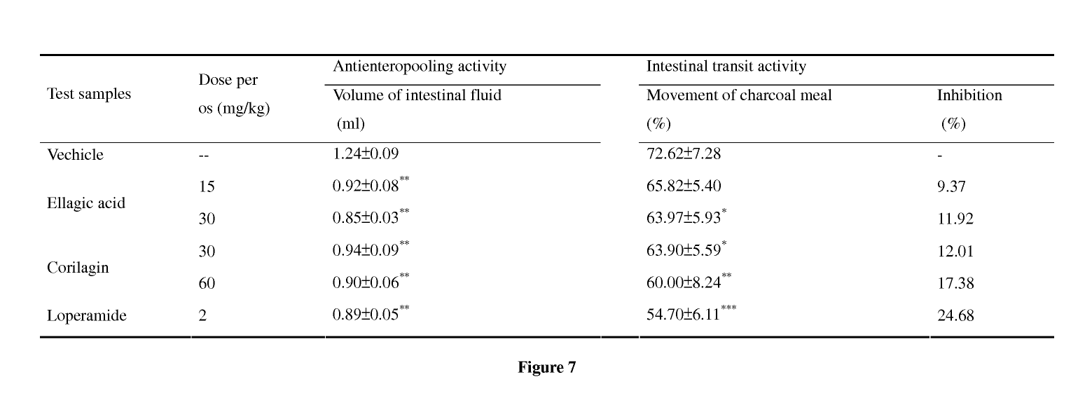

To further access the anti-diarrheal activities of those two tannins in animals, the concentrations of ellagic acid (Formula 1) and corilagin (Formula 2) in P. chinense were quantitatively analyzed and thereby to determine dosages of ellagic acid (Formula 1) and corilagin (Formula 2) used in mice. As shown in FIG. 3, 2.39 mg of corilagin and 1.04 mg of ellagic acid were examined in each gram of raw material of P. chinense. Data shows that the ethanol extract of P. chinense at dosage of 2.355 g/kg and 4.710 g/kg can significantly suppress castor oil- and magnesium sulfate-induced diarrhea in mice. Therefore, corilagin at dosages of 30 mg/kg and 60 mg/kg, and ellagic acid at dosages of 15 mg/kg and 30 mg/kg were determined to use for evaluation of their anti-diarrheal activities. As a result, both two tannins can produce significantly inhibitory effects in mouse models of castor oil- and magnesium sulfate-induced diarrhea, and ellagic acid at dosage of 30 mg/kg and corilagin at dosage of 60 mg/kg exhibited comparable anti-diarrheal activities as loperamide at dosage of 2 mg/kg (As showed in FIG. 6). Subsequently, the antienteropooling assay and gastrointestinal transit test were performed to test their effects on the gastrointestinal motility and fluid secretion. As showed in FIG. 7, the intestinal fluid volumes of the mice (from the pylorus to the caecum) is significantly reduced by corilagin both at the doses of 30 and 60 mg/kg, and ellagic acid both at the doses of 15 and 30 mg/kg as well, compared to control group. In the gastrointestinal motility test, corilagin at the doses of 15 and 30 mg/kg and ellagic acid at the doses of 30 mg/kg can significantly delay the intestinal transit of charcoal meal in mice, to exhibit a good ability to inhibit intestinal motility.

### INDUSTRIAL APPLICABILITY

The present invention discloses the utility of factions and compounds from Polygonum chinense Linn in anti-diarrhea treatment. The invention also includes their preparation and application method for anti-diarrhea treatment.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What is claimed is:

1. A composition for prevention and treatment of diarrhea and related diseases in a subject in need thereof comprising a therapeutic effective amount of bioactive fractions, compounds or extract from a herb from the family of Polygonaceae, a compound of formula (1) or (2), salts, derivatives thereof, or a combination thereof:

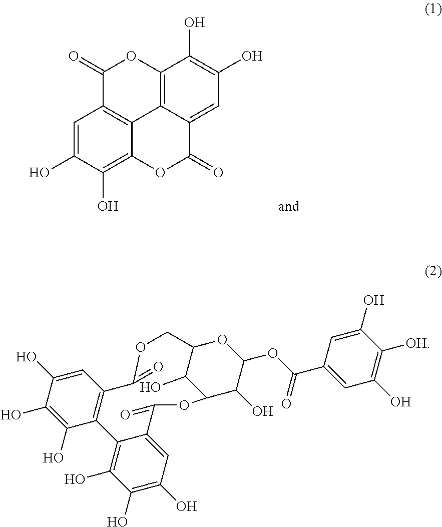 [SMILES: O=c1oc2c(O)c(O)cc3c(=O)oc4c(O)c(O)cc1c4c23 ; "and" ; O=C(OC1OC2COC(=O)c3cc(O)c(O)c(O)c3-c3c(cc(O)c(O)c3O)C(=O)OC(C2O)C1O)c1cc(O)c(O)c(O)c1]

2. The composition of claim 1, wherein the composition comprises bioactive fractions, compounds or extract from a herb from the family of Polygonaceae, the composition is orally administered to the subject in need thereof at a dosage of no less than 1.117 gram/kilogram body weight/day.

3. The composition of claim 2, wherein the dosage is 2.355 gram/kilogram body weight/day.

4. The composition of claim 2, wherein the dosage is 4.710 gram/kilogram body weight/day.

5. The composition of claim 1, wherein the compound is formula (1), and the composition is orally administered to the subject in need thereof at a dosage of no less than 15 milligram/kilogram body weight/day.

6. The composition of claim 5, wherein the dosage is 30 milligram/kilogram body weight/day.

7. The composition of claim 1, wherein the compound is formula (2), and the composition is orally administered to the subject in need thereof at a dosage of no less than 30 milligram/kilogram body weight/day.

8. The composition of claim 7, wherein the dosage is 60 milligram/kilogram body weight/day.

9. The composition of claim 1, wherein the herb is Polygonum chinense Linn.

10. The composition of claim 1, wherein the subject is a human.

11. The composition of claim 1, wherein the diarrhea related diseases comprising enteritis, dysentery, inflammatory bowel disease, irritable bowel syndrome, irritable pouch syndrome.

12. A method for prevention and treatment of diarrhea and related diseases comprising administering the composition of claim 1 to a subject in need thereof.

13. A method of preparing bioactive fractions or compounds from a herb of Polyonaceae family for treatment of diarrhea and related diseases comprising
extracting a viscous ethanol extract from the herb of Polyonaceae family, for at least two times,
evaporating the viscous ethanol extract under a reduced pressure to remove the ethanol and to concentrate the extract;
partitioning the viscous ethanol extract using n-butanol which results in a n-butanol fraction and a remainder aqueous fraction;
combining the n-butanol fraction and the aqueous fraction to form a combination fraction,
fractioning the combination fraction using a macroporous adsorption resins column by first dissolving the combination fraction in hot water and then adding slowly into a pretreated macroporous adsorption resins column;
after reaching adsorption equilibrium, resins is then eluted with a solution of a predetermined concentration to produce a dissolved fraction of the extract;
concentrating and vacuum-drying the dissolved fraction to produce the bioactive fractions or compounds from the herb.

14. The method of claim 13, wherein the herb is Polygonum chinense Linn.

15. The method of claim 13, wherein the viscous ethanol extract is extracted using six-fold 75% EtOH for 2 hours each time.
